(19) United States
(12) Patent Application Publication
(10) Pub. No.: US 2009/0104206 A1
(43) Pub. Date: Apr. 23, 2009

(54) Bone microenvironment modulated migraine treatments

(76) Inventors: Mark Zamoyski, San Jose, CA (US); Justin John Zamoyski, San Jose, CA (US)

Correspondence Address: Mark Zamoyski, 988 Foothill Drive, San Jose, CA, 95123-5301, US

(21) Appl. No.: 11/975,465

(22) Filed: Oct. 19, 2007

(51) Int. Cl. A61K 38/23; A61K 31/4535; A61K 31/135; A61K 31/66; A61K 31/663; A61K 31/675; A61K 33/24; A61K 39/395; A61P 25/06

(52) U.S. Cl. 424/158.1; 514/12; 514/324; 514/648; 514/108; 514/94; 514/89; 424/650

(57) ABSTRACT

Novel etiology and pathogenesis of premenstrual headache and premenstrual migraine are presented and novel treatment methods are provided. Present invention identifies how declining estrogen results in a transient elevation in extracellular calcium concentrations via osteoclast upregulation. The elevated extracellular calcium pathogenesis is then traced, from bone to brain, and includes depolarization of nerves, hyperactive neurotransmitter release, and hyperactive muscle contractility. Treatment methods are provided that target the earliest steps of the underlying etiology, in order to provide the most efficacious treatment possible. The treatment methods presented include use of compounds such as calcitonin and SERMs such as raloxifene.

### CROSS REFERENCE TO RELATED APPLICATIONS

Not Applicable.

### STATEMENT REGARDING FEDERALLY SPONSORED RESEARCH

Not Applicable.

### INCORPORATION-BY-REFERENCE OF MATERIAL SUBMITTED ON A CD

Not Applicable.

### BACKGROUND OF THE INVENTION

1. Field of the Invention

The invention relates to composition and methods for the treatment of symptoms related to the drop in estrogen levels prior to and during menstruation. More specifically, present invention provides novel treatment methods for premenstrual headaches, premenstrual migraines, and primary dysmenorrhea (menstrual cramps). Present invention discloses novel pathways that are responsible for the these symptoms and provides novel treatment methods based on these disclosures.

2. Description of Related Art

Premenstrual syndrome (PMS) is a very broad term that was coined in 1931, yet there is no clear consensus on a definition of the syndrome. Several hundred symptoms have been attributed to PMS and more than 80 PMS treatments have been proposed over time. The etiology of PMS remains undefined, although an abundance of prior art theories, with varying degrees of supporting evidence, exist.

There is a lack consensus as to what is encompassed by PMS. Some studies acknowledge “premenstrual headaches” (Moline and Zendell, Evaluating and Managing Premenstrual Syndrome, 2000, Medscape). Other studies categorize migraine headaches as “common disorders that may co-occur with PMS” (Ellen W. Freeman, “Epidemiology and Etiology of Premenstrual Syndromes”). “Dysmenorrhea is not PMS” according to the Freeman interpretation.

To avoid potential ambiguities, premenstrual conditions, as used in present application, are defined as conditions that begin to manifest during the period of declining estrogen levels prior to the start of menses, or early into menses, and that typically resolve at a point in time prior to the end of menses.

Three representative conditions specifically addressed under present invention are 1) premenstrual headaches, 2) premenstrual migraines, and 3) primary dysmenorrhea (menstrual cramps).

Premenstrual Headaches: For purposes of present invention, premenstrual headaches are meant to loosely refer to the following set of symptoms, as described by one sufferer: The headache starts the day before the start of menstrual bleeding and lasts until the start of menstrual bleeding. The headache first manifests as a low level headache and ramps up over several hours into persistent, intense pain that in not at the very back or very front of the head and is accompanied by a hypersensitivity to sound (but not to light). The headache may be accompanied by nausea and irritability. The sufferer prefers a dark, quiet room and going to sleep, as the headache is gone by the next morning at the start of menstruation.

Premenstrual Migraines: Premenstrual migraines are pulsating in nature, are often one sided, and may be more focused toward the front of the head. Migraines commonly occur before and during menstruation and may last from several hours to three days. Migraines have been associated with irritation of the trigeminal nerve (in the face), a spreading depolarization in brain, low serotonin levels in the brain, and vasoconstriction in the brain.

Primary dysmenorrhea: Primary dysmenorrhea usually begins a few hours before the start of menstrual bleeding and may continue for a few days. The pain is usually described as being in the lower abdomen, possibly radiating to the thighs or lower back. Women with primary dysmenorrhea have increased activity of the uterine muscle with increased contractility and increased frequency of contractions. Elevated levels of prostaglandins are typically observed.

Premenstrual Headaches:

Prior art attention to premenstrual headaches is minimal, and prior art treatment methods are minimal. The entire Medscape article on managing premenstrual syndrome (Moline and Zendell, “Evaluating and Managing Premenstrual Syndrome”, 2000, Medscape) has only a single sentence relating to treatment of premenstrual headaches which reads: “Women with premenstrual headaches should try any of the common nonprescription analgesics (aspirin, acetaminophen, ibuprofen) at the onset of the headache.”

Premenstrual Migraines:

Prior art has given considerably more attention to premenstrual migraine headaches and numerous observations and theories about both migraines and premenstrual migraines exist.

One of the first theories to explain migraines was the classic theory of vasoconstriction/vasodilation—more specifically that migraines were caused by constriction of blood vessels in the brain, followed by dilation. Brain studies during migraine have shown that blood flow to the brain is abnormal.

The theory of hyper excitability built on the idea of vasoconstriction/vasodilation by adding that migraine sufferers were extra susceptible to normal triggers, such as stress. During periods of excitability, more calcium flows from extracellular fluid to intracellular space, resulting in vasoconstriction. This theory was bolstered by studies that calcium channel blockers could prevent migraine.

Irritation of the trigeminal nerve has also been implicated in migraines. Activation of the trigeminal nerve by compounds such as nitroglycerine or capsaicin triggers migraines, lending credence to the involvement of the trigeminal nerve in migraine headaches.

A spreading area of depolarization in the cortex has also been associated with migraines, which may begin 24 hours before an attack, with the onset of the headache occurring around the time of the largest area of the brain is depolarized.

Serotonin has also been implicated in migraines, as serotonin levels in the brain are low during migraines. This theory is bolstered by the fact that serotonin agonists, such as triptans, can provide pain relief.

Although no single theory exists under prior art to explain migraines, numerous treatments exist, that provide varying degrees of relief. Migraine medications include serotonin agonists, nonsteroidal anti-inflammatory drugs, combinations of over the counter pain killers, ergot alkaloids, corticosteroids, botox injections, opiate analgesics, lidocaine applied in the nasal cavities, magnesium, butterbur root, feverfew, riboflavin (vitamin B 2), coenzyme Q10, and S-adenosyl-L-methionine.

Menstrual migraines are more specifically tied to the ovulation cycle, and are triggered during declining estrogen levels, although some women are thought to suffer migraine from the progesterone decline.

A comprehensive synopsis of prior art work related to ovarian hormones and the pathogenesis of menstrual migraine is contained in the Martin and Behbehani article enclosed under IDS (Vincent T. Martin, M D; Michael Behbehani, PhD, “Ovarian Hormones and Migraine Headache: Understanding Mechanisms and Pathogenesis—Part I”, ©2006 Blackwell Publishing, Medscape Jan. 26, 2006). Migraines are 3 times as common in women than in men and migraine attacks are commonly triggered by declines in serum estrogen levels. Accordingly, prior art menstrual migraine research is focused on ovarian hormone effects on A) serotonergic, B) noradrenergic, C) glutamatergic, D) GABAergic, and E) opiatergic systems, as disclosed in the article. The article then considers other possibilities, focusing on ovarian hormone effects on specific structures relevant to migraine headache such as meningeal arteries and the trigeminal nerve. A synopsis of the prior art synopsis is provided for reference:

A) Serotonergic. Serotonin (5-hydroxytryptamine; 5-HT) is a neurotransmitter that acts on seven distinct families of 5-HT receptors (5-HT1 to 5-HT7) and each receptor has multiple subtypes. Under prior art “Substantial evidence exists to suggest that the serotonergic system is important in the pathogenesis of migraine headache. A positron emission tomography (PET) study demonstrated increased serotonin synthesis capacity throughout all regions of the brain in migraine patients as compared to controls. Medications which are agonists of the 5-HT1B, 5-HT1D, and 5-HT1F receptors are efficacious abortive treatments for migraine headaches” (Martin and Behbehani).

Prior art has also demonstrated that estrogen effects serotonin by three pathways. First, estrogen treated monkey showed a nine-fold increase in tryptophan hydroxylase (TPH), the rate-limiting enzyme in synthesis of serotonin. Second, the serotonin reuptake transporter (SERT) removes serotonin from the synaptic cleft to terminate serotonergic transmission. Short term estrogen treatment of monkeys decreased amounts of SERT miRNA and longer treatments led to increased amounts of SERT mRNA. Third, monoamine oxidases, the primary enzymes that degrade serotonin, were reduced in monkeys receiving estrogen. Less compelling evidence suggests estrogen/progesterone combinations may modulate gene expression and binding potentials of serotonin receptors.

B) Noradrenergic System. The Martin and Behbehani article discloses that estrogen has been shown to up-regulate production of noradrenaline by up-regulating gene expression of tyrosine hydroxylase, a rate-limiting step in the production of noradrenaline. Studies also exist to show that estrogen may effect various subtypes of adrenoreceptors. The article also discloses that noradrenaline levels are decreased in migraineurs during headache free periods, suggestive of a state of chronic sympathetic hypofunction. Other studies imply that estrogen alone reduces central sympathetic activity, but the addition of progesterone may actually increase sympathetic tone.

C) Glutamatergic System. Glutamic acid is the major excitatory neurotransmitter in the central nervous system (CNS). The studies reviewed by Martin and Behbehani indicate that estrogen is a significant facilitator of the glutamatergic system and that certain effects can be attenuated by addition of progesterone.

D) GABAergic System: GABA is the major inhibitory neurotransmitter in the CNS. In vitro studies indicate that both estrogen and progesterone modulate GABAergic neurons. In vivo, women with premenstrual dysphoric disorder (PMDD) demonstrated increased cortical GABA during luteal phase (when both estrogen and progesterone levels are high) when compared to follicular phases (when estrogen is high but progesterone is low). The control group showed the opposite results with higher GABA levels in the follicular phases than the luteal phases.

E) Opiatergic System: The opiatergic system is important for pain control and regulation of reproductive behavior. Estrogen has been shown to increase levels of spinal cord enkephalin and enhance neuronal responsiveness of certain opioid receptors.

The article also covers other prior art theories by reviewing effects of ovarian hormones on specific structures relevant to migraine headaches.

Trigeminal Nerve: The trigeminal nerve is know to be involved in migraine headaches. The effects of ovarian hormones on the trigeminal nucleus caudalis (TNC) have been well studied. Animal model data show greater response magnitude and response duration of TNC neurons (i.e. enhanced sensitivity) is observed when estradiol and progesterone levels are high. It should be noted this is inconsistent with a premenstrual migraine, as falling levels of both hormones would predict reduced sensitivity of the TNC. However, TNC hypersensitization is consistent with falling estrogen levels under the novel etiology provided in present invention.

Brainstem Nuclei: The Martin and Behbehani article also postulate that ovarian steroids could potentially modulate neurotransmission within the brainstem nuclei to account for the increased blood flow to the dorsal pons observed on PET scans during spontaneous migraines.

Autonomic Nervous System: Estrogen alone reduces central sympathetic activity, reducing heart rate and sympathetic tone, wile increasing parasympathetic tone. Addition of progesterone increases sympathetic tone. Chronic sympathetic hypofunction during headache-free period has been suggested in 10% to 15% of migraineurs.

Vascular Endothelium: Estrogen produces vasodilation through endothelium-dependent and non endothelial dependent mechanisms. The article suggests TNC sensitization by vasodilation of meningeal arteries.

Cortex: The anterior cingulate and insular cortices are activated on PET studies during a migraine attack. The article suggests ovarian steroids may modulate migraine on a cortical level.

Primary Dysmenorrhea:

Women with primary dysmenorrhea have increased activity of the uterine muscle with increased contractility and increased frequency of contractions. Cramping associated with dysmenorrhea usually begins a few hours before the start of bleeding and may continue for a few days.

Prior art dysmenorrhea treatment methods center around prostaglandin inhibition. Prostaglandin levels have been found to be higher in women with severe menstrual pain than in women who experience mild or no menstrual pain. Non-steroidal anti-inflammatory drugs (NSAIDs) that inhibit prostaglandin synthesis can provide relief and include drugs such as Naproxen, Ibuprofen, and Mefenamic Acid. However, many NSAIDs can cause gastrointestinal upset as a side effect and COX2 inhibitors are sometimes prescribed instead. Oral contraceptives are effective in preventing dysmenorrhea as they suppress ovulation and menstruation.

### BRIEF SUMMARY OF THE INVENTION

The present invention discloses a novel underlying pathogenesis that results in premenstrual conditions such as premenstrual headache, premenstrual migraine, and primary dysmenorrhea. The present invention will explain the prior art observations in context of the new pathways presented. Based on this novel disclosures, novel treatment methods are provided.

More specifically, present invention discloses estrogen's role in “reservoiring” calcium in bone during most of the ovulation cycle (via osteoclast inhibition pathways when estrogen levels are high), then releasing the reservoired calcium and growth factors prior to menstruation (via removal of osteoclast inhibition as estrogen levels drop), and the resulting transient extracellular calcium “spike” causes both neurosensory and neuromuscular hypersensitization that accounts for the spectrum of observed symptoms previously disclosed.

Accordingly, the novel treatment methods focus on managing the transient calcium spike via both transient osteoclast downregulation and other direct methods of transient calcium downregulation.

### BRIEF DESCRIPTION OF THE DRAWINGS

FIG. 1 shows estrogen levels during the ovulation cycle.

FIG. 2 shows estrogen's effect of “reservoiring” and “release” of calcium and growth factors (via osteoclast population density modulation) during the ovulation cycle

FIG. 3a shows the region of the brain where the somatosensory and auditory cortex are located and

FIG. 3b shows the mapping of peripheral sensory nerves to the somatosensory cortex.

### DETAILED DESCRIPTION OF THE INVENTION

### Overview

Prior art has focused on numerous possible pathophysiologies (previously presented) related to a set of conditions that occur prior to start of menstruation and resolve by the end of menstruation. Numerous prior art theories exist about these conditions, and in many of these theories, observations are elevated to etiology status. No single prior art theory can explain the conditions, and as such, other theories have been proposed that the conditions are caused by a collection of disorders.

In contrast, present application presents a single underlying event that occurs, which in turn effects several physiological systems, which in turn accounts for the symptoms and observations related to several premenstrual conditions. These premenstrual conditions include premenstrual headache, premenstrual migraine, and primary dysmenorrhea.

More specifically, the present invention outlines estrogen's role in “reservoiring” and “releasing” calcium and growth factors (via osteoclast population density modulation) over the course of the ovulation cycle, with a specific focus on the systemic effects of the abrupt transition that occurs prior to the start of menstruation.

The underlying pathogenesis is then clinically corroborated by both the actual human symptoms described and by observations from the various brain scan technologies used.

Novel treatment methods are then provided that target the earliest steps in the pathogenesis.

### The Bone Micro Environment

Because the underlying pathogenesis of present invention starts with estrogen's effect on the bone micro environment, a brief background of the bone micro environment is provided for reference.

Normal bone undergoes a continual remodeling process that essentially replaces the entire skeleton every 10 years. Remodeling is mediated by two cell types, osteoclasts which dissolve bone (resorption), and osteoblasts which are the bone builders. Both cell types come together in three to four million remodeling sites scattered throughout the skeleton. During childhood and adolescence, bone formation proceeds at a faster rate than resorption. By around age 40 bone resorption begins to outpace bone formation and bone thinning begins to manifest. On average, women attain a peak bone mass that is about 5% below that of a man, so they have less “in the bank” to start with at the onset of age related bone loss. For this and other reasons, risk of osteoporosis (literally “porous bone”) is greater in women, who account for 80% of cases.

Osteoblasts (the bone building cells) secrete collagen and other bone proteins creating a matrix onto which calcium, phosphorous, and other minerals crystallize (˜90% of bone mass), which removes calcium from extracellular fluid and blood circulation. Osteoclasts (the bone dissolving cells) secrete both proteolytic and hydrolytic enzymes and hydrochloric acid that result in destruction of the bone's protein matrix, which in results in mobilization of calcium, phosphorous, and bone resident growth factors, into the extracellular fluid.

In addition to providing structural support and organ protection, bone serves as a repository of calcium and is used to maintain serum calcium concentrations. The average adult human body contains 1.3 kg of calcium of which 99% is contained in bones and teeth, 1% in cells of soft tissue, and 0.15% in the extracellular fluid. Normal serum plasma levels of calcium range from 8.0 to 10.8 mg/dl (2.2 to 2.7 mmol/L) with 40%-43% bound to plasma proteins, 5%-10% combined with anions such as phosphate and citrate to form non ionized complexes, and the remaining 40%-50% being free ionized calcium. The primary hormone responsible for increasing serum concentrations of calcium is parathyroid hormone(PTH) and the primary hormone responsible for reducing serum concentrations of calcium is calcitonin, which is produced by the parafollicular cells of the thyroid. When calcium sensors in the parathyroid gland detect low serum calcium concentrations, production of PTH is upregulated, resulting in upregulated osteoclastic activity and increased renal reabsorption of calcium. High serum calcium concentrations result in upregulated production of calcitonin, resulting in decreased osteoclastic activity and up to a 5 fold increase in renal excretion of calcium.

In addition to calcium, phosphorus and various growth factors are also stored in the bone, and are mobilized into the extracellular fluid by osteoclast activity. The calcium to phosphorous ratio in bone is 2.5 to 1 and phosphorus is involved in numerous physiological processes including transport of cellular energy via adenosine trisphosphate (ATP), phosphorous is important for key regulatory events such as phosphorylation, and phospholipids are the main structural components of cellular membranes. Phosphorous is also used in maintenance of extracellular/intracellular ion concentration gradients via transmembrane ATPase pumps. Growth factors that are stored in bone and liberated by osteoclast activity include platelet-derived growth factors (PDGF), fibroblast growth factors (FGF), insulin like growth factors (IGFs) I and II, transforming growth factor-beta (TGF-beta), endothelin 1 (ET-1), urokinase type plasminogen activators, and others. The growth factors released from bone are potent mitogens. PDGF and FGF are mitogens that stimulate progression of many cell types through the early part of the G-1 Phase and IGF-1 and IGF-2 are potent mitogens that promote cell progression through the later part of the G-1 Phase.

Osteoclasts arise through the differentiation of macrophages. Osteoclasts are regulated by several hormones including PTH from the parathyroid gland, calcitonin from the thyroid gland, estrogen, vitamin D, and growth factor interleukin 6 (IL-6). Osteoclast population density is modulated by three molecules produced by osteoblasts—two that promote osteoclast development and one that suppresses osteoclast development. The two osteoclast promoter molecules are 1) macrophage colony-stimulating factor that binds to a receptor on macrophages inducing them to multiply and RANKL (receptor activator of NF-kB ligand) that binds to a different receptor (RANK receptor) inducing the macrophage to differentiate into an osteoclast. The molecule that inhibits osteoclast formation is osteoprotegerin (OPG), which blocks osteoclast formation by latching on to RANKL and blocking its function.

Osteoclast activity is modulated by various compounds through the following pathways.

PTH interacts with its receptor on osteoblasts to upregulate production of RANKL, which upregulates macrophage differentiation into osteoclasts. Additionally, PTH increases calcium reabsorption by the renal tubules and stimulates conversion of vitamin D to its active form (calcitriol).

Calcitonin receptors have been found in osteoclasts and osteoblasts and single injections of calcitonin result in the loss of the ruffled osteoclast border responsible for resorption of bone and a marked transient inhibition of the ongoing bone resorptive process. Calcitonin also increases renal excretion of calcium by decreasing reabsorption by the kidneys and evidence exists that it reduces absorption of calcium in the gastrointestinal tract.

Estrogen has a “triple whammy” (Dr. Clifford J. Rosen, “Restoring Aging Bones”, Scientific American, March 2003) effect in inhibiting osteoclast activity by binding to osteoblasts and 1) increasing their output of OPG and 2) suppressing their RANKL production. In addition, estrogen appears to prolong lives of osteoblasts while simultaneously 3) promoting osteoclast apoptosis. As estrogen levels drop after menopause, these “brakes” on osteoclast inhibition are removed, tipping the balance in favor of osteoclast dominated bone destruction which results in osteoporosis.

Androgens also have an inhibitory effect on bone resorption, and studies suggest that this occurs through local aromatization of androgens into estrogen, however direct androgen interactions with androgen receptors(AR) related to bone remodeling have been observed in animal models.

Vitamin D is a steroid-like chemical that promotes osteoclast activity by binding to vitamin D receptors (VDR) in osteoblasts and upregulating expression of RANKL.

### Estrogen, Bone, and Extracellular Calcium Levels

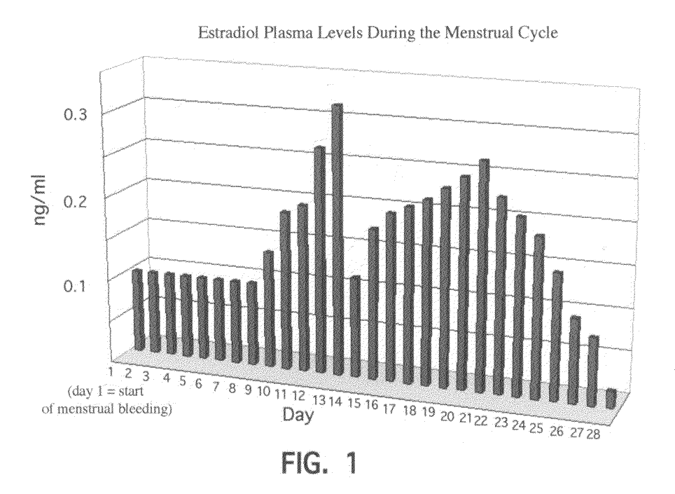

Serum estrogen levels vary throughout the ovulation cycle as shown in FIG. 1, which is excerpted from a reference text graph of ovulation hormone levels (“Biochemical Pathways”, Gerhard Michal, Wiley & Sons, 1999, page 205, FIG. 17.1-6). Beginning about 20 days prior to the start of menstrual bleeding, estrogen levels rise to double to triple the levels observed during menstruation. A few days prior to start of menstrual bleeding, estrogen levels begin to decline, with the most precipitous decline occurring the day before the start of menstrual bleeding.

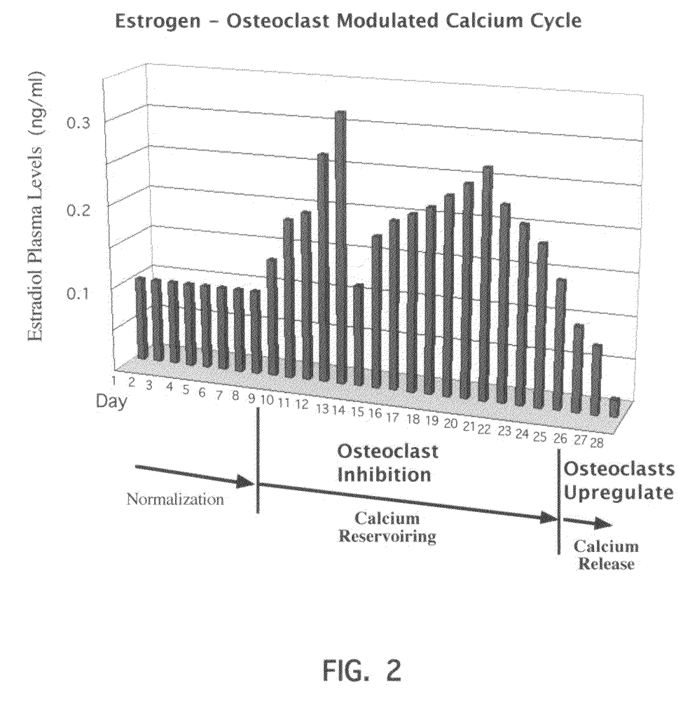

From osteoporosis research, it is known that estrogen inhibits osteoclast activity by at least 3 pathways (i.e. the “triple whammy” previously disclosed). Accordingly, elevated estrogen levels tip the balance in favor of osteoblast activity, which result in net bone building activity, which in turn includes storage of calcium and growth factors in bone. This is referred to as “reservoiring” in this application and occurs during the time estrogen levels are elevated as shown in FIG. 2 (likely in anticipation of pregnancy, as both calcium and growth factors are integral for cell growth and division via activation of the cell cycle control system). The subsequent drop in estrogen levels (in the absence of pregnancy) removes the inhibitory effects on osteoclasts, which tips the balance in favor of bone resorption activity, which in turn includes release of calcium and growth factors along the approximate timeline shown in FIG. 2.

The most precipitous decline in estrogen levels occurs a day or so prior to the start of menstrual bleeding, and accordingly the highest release of bone resident calcium would also occur around this time, hereinafter referred to as the “calcium spike”. As extracellular concentrations of calcium begin to rise, the concentrations work their way through into blood circulation, where the escalating serum concentrations activate the body's serum calcium control mechanisms (via calcitonin). Blood calcium concentrations are tightly controlled (unlike extracellular concentrations the have a greater range of variability) and renal excretion of serum calcium can increase 5 fold (provided it does not get overwhelmed) to maintain serum calcium homeostasis and osteoclasts activity is inhibited (osteoclasts lose their ruffled border that dissolves bone) to reduce the amount of calcium being mobilized from the bone into the extracellular fluid.

Although calcitonin has significant calcium lowering effects in some species, in humans, calcitonin's influence on blood calcium levels is much smaller. Human calcitonin is not used for management of hypercalcemia, instead salmon calcitonin is used, as it is around 40-50 times more potent than human calcitonin and has a longer duration of action. Despite the higher potency of salmon calcitonin, its effects on reducing serum calcium levels are often inadequate to manage conditions such as hypercalcemia of malignancy, requiring the use of even more potent drugs such as bisphosphonates that induce osteoclast apoptosis.

Accordingly, the naturally weak human calcitonin based serum Ca²⁺ downregulation system would likely be playing catch-up with the progressively elevating Ca²⁺ release caused by the premenstrual estrogen decline. Furthermore, the rising extracellular calcium concentrations would not have the direct benefit of renal clearance that blood circulation does, and there would be much sharper escalations in extracellular calcium concentrations than in blood. This is important to note, as extracellular calcium concentrations (and more specifically concentrations surrounding nerve and muscle membranes) are of primary importance to present invention, and not blood concentrations.

The worst peak in calcium concentrations would occur the day prior to start of menses (i.e. as a result of the sharp premenstrual estrogen drop), after which point calcium levels would start normalizing as estrogen levels normalized and calcitonin would have finally caught up and eventually managed to work its way back to reducing extracellular calcium concentrations.

### Extracellular Calcium and the Nervous System

Transiently increased extracellular Ca²⁺ levels effectively “hyper sensitize” nerves by three pathways described below.

The fundamental task of a neuron is to receive, conduct, and transmit signals. Neurons can be classified by function into sensory neurons, motor neurons, or interneurons, however they all have the same overall structure. Neurons have a spherical central cell body (soma) that contains the typical organelles found in all cells, branching dendrites on one side to receive signals, and a long axon on the other side for transmitting information. The axon commonly divides into many branches at its far end so it may pass the message to many target cells simultaneously. A signal travels along the neuronal membrane as an electrical pulse until it reaches the end of the axon, where typically the electrical pulse results in neurotransmitter release across the synapse, which in turn results in an electrical pulse being induced in the next neuron.

Neurons contain ion channels that maintain a balance between potassium, sodium, and chloride so that the resting membrane potential inside of the neuron is around −85 mV relative the outside of the cell (ranges from −30 mV to −100 mV depending on cell type). The cell membrane acts as a capacitor, storing charge separated by the thickness of the membrane, and has a typical capacitance of about 1μ Farad per square centimeter. Changes to the membrane potential are called “depolarizing” if they make the inside of the cell less negative or “hyperpolarizing” if they make the inside of the cell more negative. Electrical impulses that travel along the neuron are called action potentials and are transient perturbations in the membrane potential. Action potentials are conducted in a all-or-none manner and for an action potential to be generated the input signal must depolarize the neuron by more than its “threshold” membrane potential. As an example, for the −85 mV resting membrane potential neuron above, the threshold voltage is around −70 mV, meaning that the input signal must depolarize the membrane by at least 15 mV to generate a nerve impulse (i.e. action potential).

Changing the extracellular or intracellular concentrations of ions changes the resting membrane potential. Depolarizing concentrations (i.e. that make the inside of the cell less negative) bring the resting membrane potential closer to the threshold potential, and consequently the neuron requires a smaller input voltage to trigger an action potential. Polarizing concentrations (those that make the inside more negative) move the resting membrane potential farther away from the threshold potential and result in a larger input signal being required to trigger an action potential.

A traveling nerve impulse opens voltage gated Na+ channels and K+ channels, which allow Na+ to flow into the cell and K+ to flow out of the cell, passively along their respective electrochemical gradients. Both the Na+ channels and K+ channels are rapidly inactivated by a “ball and chain” amino acid complex that rapidly plugs the respective channels. Potassium (K) is the most significant ion in impulse transmission because of the large disparity between the extracellular and intracellular concentrations. Typical extracellular concentrations potassium and sodium are about 3 mM of K+ and 117 mM of Na+ and the typical intracellular concentrations are about 90 mM of K+ and 30 mM of Na+. The 30 fold concentration gradient disparity of K+ ( i.e. 90/3) overwhelms the 4 fold gradient disparity of Na+ (i.e. 117/30).

The resting (equilibrium or E) membrane potential for a given ion (e.g. potassium) can calculated using the Nernst equation:

Eₖ=RT/zF(ln([K]ₒ/[K]ᵢ))

where:

Ek is the equilibrium (or resting) membrane potential for potassium

R is the gas constant (8.31 joules/mole/° K)

T is the absolute temperature (Kelvin=273+° C.)

z is the valence of the ion (+1 for potassium)

F is the Faraday constant (amount of charge on a mole of ions, 96,500 coulombs/mole)

Ko is the outside (extracellular) concentration of potassium (in mM) and

Ki is the inside (intracellular) concentration of potassium

At room temperature (20° C.=293° K.) and for potassium:

RT/zF=(8.31)(293)/(+1)(96,500)=0.02523 V=25 mV

and for concentrations of 3 mM outside the cell and 90 mM inside the cell:

Eₖ=(25 mV)(ln([K]ₒ/[K]ᵢ))=(25 mV)(ln 3/90)=(25 mV)(−3.4)=−85 mV

The effect of elevating extracellular concentrations of positive ions can be seen from the Nernst equation. Increasing extracellular concentration of the positive ion K+ results in a more positive resting membrane potential, which is by definition depolarizing, and brings the resting membrane potential closer to the threshold potential. This means a smaller input signal voltage is required to trigger the “all-or-none” action potential.

As an example, as extracellular concentrations of K+ are raised to 4 mM, the resting membrane potential becomes more positive:

Eₖ=(25 mV)(ln(4/90))=(25 mV)(−3.11)=−78 mV

Using the −70 mV threshold voltage, the input voltage required to initiate an action potential is now only 8 mV versus 15 mV. Applicants refer to this as “neuronal membrane hypersensitization” in present application.

The actual resting membrane potential is a summation of all ions that are permeable and can be more precisely calculated using the Goldman Hodgkin Katz equation (GHK) for computing the resting membrane potential:

$${V}_{m} = 58 log \frac{( pk [ K ] o + pNa [ Na ] o + pCl [ Cl ] i )}{( pk [ K ] i + pNa [ Na ] i + pCl [ Cl ] o )}$$

Where:

Vₘ is the resting membrane potential.

pI is the permeability of an ion.

[I]o is the extracellular concentration of an ion.

[I]i is the intracellular concentration of an ion.

The GHK equation above does not include Ca²⁺, however, since calcium ions are permeable through the sodium-calcium exchanger, for precise calculations, Ca²⁺ would need to be included in the above GHK equation.

Alternatively, the Nernst equation provides a good way of estimating an individual ion's contribution to the overall resting membrane potential.

From the Nernst equation, we can see that increasing extracellular concentrations of positive ions, relative to intracellular concentrations of positive ions, is a depolarizing change. Accordingly, elevating extracellular Ca²⁺ levels relative to intracellular Ca²⁺ levels is a depolarizing event that would lead to neuronal membrane hypersensitization (i.e. reducing the magnitude of the input signal required to initiate an action potential).

Neuronal intracellular calcium (Ca²⁺) levels are kept low as calcium is a signaling molecule within a neuron (used for neurotransmitter release at the synapse). Calcium ATPase pumps in the cell membrane and in the membranes of intracellular organelles pump calcium out of the cytoplasm. Extracellular concentrations of Ca²⁺ can range from 1 to 2 mM (Molecular Biology of the Cell, third edition, p. 508). However, intracellular concentrations are kept very low and do not increase proportionately relative to extracellular increases. Studies of mammalian brain nerve cells showed that as extracellular concentration of Ca²⁺ were raised from 1 mM to 2 mM, the intracellular concentrations only rose from 130 nM to 160 nM, respectively (D. A. Nachshen, J. Physiol. June 1985; 363: 87-101, FIG. 1B on page 90, provided under IDS). Accordingly, for a 100% increase in extracellular concentrations of Ca²⁺, the intracellular concentrations only rise 25%.

From the above information we can approximate the amount of depolarization that would occur across the range of 1 mM to 2 mM of extracellular Ca²⁺. Using the Nernst equation and the change in the E_{Ca} between the 2 nM and 1 nM levels would provide the amount of depolarization in mV that could be expected (per 1 mM) over this range (i.e. E_{Ca}@2 mM−E_{Ca}@1 mM=net change in resting membrane potential from a 1 mM change in extracellular Ca²⁺ concentrations), or:

ΔE_{Ca} per 1 mM increase in [Ca]ₒ=E_{Ca}@2 mM−E_{Ca}@1 mM

For calcium, RT/zF=(8.31)(293)/(+2)(96,500)=12.6 mV and the

$$\begin{matrix}Δ {E}_{Ca} = ( 12.6 mV ) ( ln ( 2 / .000160 ) ) - ( 12 mV ) ( ln ( 1 / .000130 ) ) \\ = ( 12.6 mV ) ( 9.43 ) - ( 12.6 mV ) ( 8.948 ) \\ = + 6.12 mV\end{matrix}$$

Accordingly, the increase in extracellular Ca²⁺ concentrations from 1 mM to 2 mM would make the resting membrane potential more positive by around 6 mV. In our previous example, this would reduce the resting membrane potential from −85 mV to −79 mV, which in turn would reduce the amount of input stimulus required to trigger a nerve impulse from 15 mV to 9 mV.

This neuronal membrane hypersensitization disclosed above is the first mechanism by which rising calcium ion concentrations would affect the nervous system.

The second mechanism is calcium related neurotransmitter release, as it relates to both sensory receptor transduction signaling and synaptic gap signal transmission.

As a nerve impulse reaches the synapse, voltage gated Ca²⁺ channels open which allow an inrush of Ca²⁺ to enter the pre synaptic cell, along its electrochemical concentration gradient. Neurotransmitter is stored in vesicles and Ca²⁺ causes the vesicles to fuse with the cell membrane, releasing the neurotransmitter by exocytosis into the synaptic cleft. The neurotransmitter binds to and opens transmitter-gated ion channels on the post synaptic cell, which triggers a depolarization in the post synaptic cell, triggering an action potential if sufficient depolarization occurs. The extent of the depolarization of the post synaptic cell is graded according to how much neurotransmitter is released at the synapse and how long it persists there (Molecular Biology of the Cell, third edition, p. 536).

As extracellular Ca²⁺ levels increase from 1 mM to 2 mM, not only does the absolute amount of molecules available to rush in through the voltage gated channels double, but the concentration gradient (i.e. the driving force for the inrush) increases from being 7,672 times greater on the outside at 1 mM ( i.e. 1 mM/130 nM) to being 12,500 times greater on the outside at 2 mM ( i.e. 2 mM/160 nM). Accordingly, the much larger amount of Ca²⁺ entering the pre synaptic cell during the transient period when the voltage gated channels are open would result in much greater release of neurotransmitter. Since depolarization of the post synaptic cell is graded and related to the amount of neurotransmitter released, as previously disclosed, the effect of rising extracellular Ca²⁺ levels would also be “hypersensitization of synaptic gap transmission” via greatly “upregulated neurotransmitter release” from the pre synaptic cell combined with the “neuronal membrane hypersensitization” in the post synaptic cell (i.e. the depolarization per the Nernst equation). Accordingly, rising extracellular calcium concentrations would have a direct “double whammy” effect on nerves.

A third mechanism, known as posttetanic potentiation (PTP), can also cause over-excitation in brain neurons form increased transmitter release related to the inability of the neurons to clear the Ca²⁺ inrush in a timely manner. PTP occurs normally in response to a long high frequency train of action potentials (e. g. 100 action potentials pre second for 15 seconds). A tetanic train of potentials will cause a large increase in the concentration of cytoplasmic calcium that cannot be readily cleared. This calcium will then travel down the mitochondrial calcium uniporter to increase the mitochondrial calcium levels. After the tetanic train, cytoplasmic calcium will be pumped out of the cell and when the cytoplasmic level is low enough, calcium from the mitochondria enters the cytoplasm. While this happens, any action potential that occurs in this time frame, will cause more transmitter release, because of the elevated intracellular calcium levels (i.e. PTP). In other word, the higher levels of intracellular calcium result in larger amounts of neurotransmitter being released in response to neuronal depolarization. This increases the strength and duration of the signal in the brain for a given level of stimulus. Elevated levels of extracellular calcium could be expected to exacerbate PTP type un-cleared intracellular calcium levels, as well as reduce the frequency and duration of the input train of action potentials required to trigger this condition.

The term neuronal “hypersensitization” is hereinafter used to describe the effect of elevated calcium levels on nerves (i.e. via 1) neuronal membrane depolarization, 2) upregulated neurotransmitter release at synapses, and 3) PFP mechanisms).

Extracellular Ca²⁺ levels also have a direct effect on muscle tissue, discussed below.

### Extracellular Calcium and Muscles

Transiently elevated extracellular calcium levels would increase muscle contraction by two pathways.

The first relates to nerves and the neuromuscular junction. Muscle contraction is triggered by a nerve impulse traveling down a neuron which is then converted to a release of the neurotransmitter acetylcholine at the synapses where the neuron meets the muscle. Enhanced neurotransmitter release results when extracellular Ca²⁺ levels are high, by the voltage gated Ca²⁺ channel pathways previously disclosed above. Accordingly, more acetylcholine is released at the neuromuscular junction, causing a greater post synaptic depolarization.

The second pathway relates to extracellular Ca²⁺ concentration's direct effect on muscle contraction. The release of the neurotransmitter acetylcholine described above causes the muscle to depolarize via neurotransmitter gated channels. The depolarization spreads along the muscle surface and the T-tubules that run along the surface of the muscle fibers. The depolarization opens voltage gated Ca²⁺ channels in the T-tubule surface that allows Ca²⁺ from the extracellular fluid in the T-tubule to enter the the sarcoplasmic reticulum. The inrush of Ca²⁺ into the sarcoplasmic reticulum activates the “sarcoplasmic reticulum calcium release channels” (SRCaRCs), which in turn release Ca²⁺ into the fluid around the myofibrils. The released Ca²⁺ allows the muscle to contract by removing the tropomyosin block between actin and myosin, triggering cross-bridge formation by enabling myosin to bind to actin.

With an increase in the extracellular calcium concentration, there will be a larger release of Ca²⁺ from the T-tubules, which in turn will activate more SRCaRCs and the release of more Ca²⁺ onto the myofibrils, which in turn will cause greater cross-bridge formation and muscle contraction.

### Caveats/Extensions

It should be noted that the above analysis is related to acute (i.e. transient or short term) rising calcium concentrations, and would not apply to chronic (i.e. persistent or long term) elevated calcium levels, where continual or excessive nerve firing may deplete neurotransmitter availability. Neurotransmitter is degraded after release into the synapse and new neurotransmitter must be continually synthesized in the cytosol of the neuron. Excessive neurotransmitter release, and hence excessive neurotransmitter degradation, could result in depletion of neurotransmitter availability, which in turn would result in effects such as reduced muscle contractility.

The contributory role, if any, of the estrogen-osteoclast mediated release of phosphorus and growth factors is less clear. As an example, Ca2+ ATPase pumps (used for concentration gradient maintenance) account for 90% of the membrane protein of the sarcoplasmic reticulum of muscle cells, however no clear evidence could be found that increasing phosphorous levels would effect the number or function of these pumps. The role of potassium and growth factors would also be moot, as methods of present invention downregulate osteoclast activity, hence also downregulating phosphorous and growth factor release. Furthermore, the transient calcium spike effects described above adequately account for the different symptoms and observations as discussed in detail below.

### Clinical Corroboration

Having defined the novel pathogenesis of estrogen modulated nerve and muscle hypersensitization via osteoclast modulated Ca²⁺ release, we can view the symptoms and imaging data to see if they corroborate or contradict the underlying pathogenesis presented.

Clinical Corroboration—Premenstrual Headaches:

Hypersensitivity to Sound: The hypersensitivity to sound is consistent with the neuronal hypersensitization pathways disclosed above. Auditory stimulus would be abnormally amplified in the presence of elevated extracellular Ca²⁺ levels. Accordingly, this symptom is consistent the presented pathogenesis.

No hypersensitivity to light: The absence of hypersensitivity to light is also important as it also corroborates the pathogenesis presented. While hair and somatosensory receptor cells depolarize when stimulated, human light sensing rod cells function in an opposite manner and hyperpolarize in light.

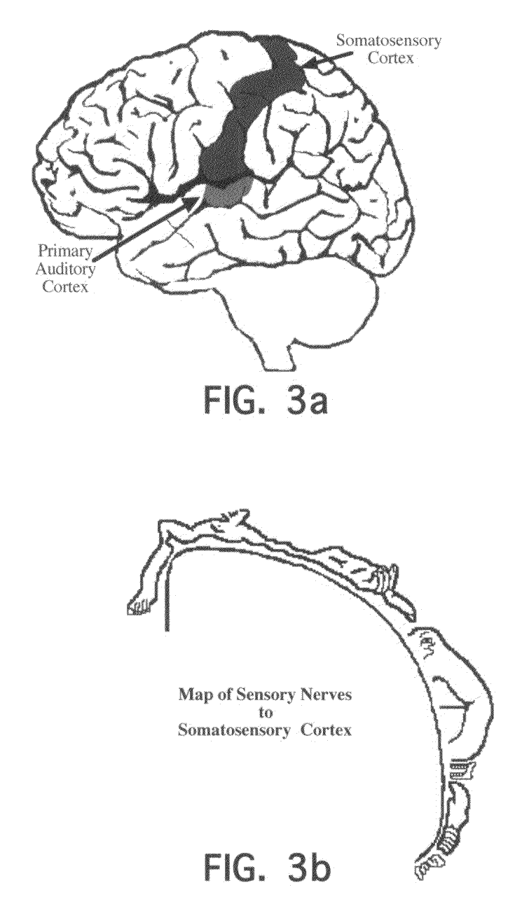

Location of Headache: The location of the headache in the center of the head, and not pronounced at the front or back, is consistent with the location of the somatosensory cortex in the brain as shown in FIG. 3a (darker shaded area). The auditory cortex is just below the somatosensory cortex as shown in FIG. 3a (lighter shaded area). The continual signals from hyper sensitized sensory neurons would effectively result in a sensory “overload” in this region of the brain. Accordingly, the location of the headache is also consistent with the pathogenesis presented.

Desire for quiet, dark room and sleep to ameliorate the symptoms: This is consistent with the pathogenesis presented from two standpoints. First, the “sensory deprivation” provided by this environment would function to counteract the neuronal hypersensitization by depriving nerves of any stimulus at the very front end of the process. Second, the absence of light results in the downregulation of vitamin D synthesis (i.e. the sunshine vitamin) which in turn results in downregulation of serum Ca²⁺ levels as previously disclosed (i.e. preventing vitamin D interaction with vitamin D receptors in osteoblasts prevents RANKL production via this pathway, which in turn downregulates osteoclast population density and the related release of Ca²⁺ from bone).

Clinical Corroboration—Premenstrual Migraines:

Trigeminal Nerve: Migraines have been associated with irritation of the trigeminal nerve. Migraines are triggered experimentally by compounds such as nitroglycerine which activates trigeminal nociceptors. The trigeminal nerve conveys sensory information for the face and much of the head. FIG. 3b shows the disproportionately large area of somatosensory cortex that maps to the face and head. The “irritation” of the trigeminal nerve is consistent with the hypersensitization of the trigeminal nerve system predicted by the elevated Ca²⁺ levels as previously presented ( i.e. Ca²⁺ mediated membrane depolarization and Ca²⁺ voltage gated channel mediated amplified neurotransmitter release).

Spreading Depolarization: A spreading area of depolarization in the cortex has been associated with migraines, which may begin 24 hours before an attack, with the onset of the headache at the time of the largest area of the brain is depolarized. This is consistent with the pathways presented under present invention, as the cortical depolarization would be predicted by the three pathways disclosed related to neuronal depolarization/hypersensitization from elevated Ca²⁺ levels.

Vasoconstriction: The hypersensitization of nerves and enhanced muscle contractions related to elevated Ca²⁺ levels could also be expected to result in hyper vasoconstriction. Nerve signals to vascular smooth muscle cell would be amplified by 1) Ca²⁺ motoneuron membrane depolarization, 2) motoneuron neurotransmitter release would be amplified via the voltage gated Ca²⁺ channels at the synapse, and 3) the force of vascular smooth muscle contraction would be amplified in the muscle tissue itself via the amplified Ca²⁺ release into the muscle fibers and the resulting amplified actin/myosin interactions. Vasodilation is consistent with eventual neurotransmitter depletion.

Low Cerebral Serotonin: As previously disclosed, low serotonin levels are observed during migraines and PET scans showed increased serotonin synthesis capacity in migraine patients. Serotonin is synthesized from the essential amino acid tryptophan via tryptophan hydroxylase and is degraded into 5-hydroxytryptophol or 5-hydroxyindoleacetic acid via the enzyme MAO. Pathogenesis of present invention is consistent with low serotonin levels during migraine as excessive firing from hypersensitized nerves throughout the body and in the brain would result in excessive release of serotonin at the synaptic clefts, which in turn would result in excessive degradation of serotonin. The serotonergic response is terminated by reuptake into the pre synaptic axon terminal and “The major pathways for the degradation of serotonin are reuptake into the nerve and degradation by MAO” (Neuroscience In Medicine, Second Edition, Humana Press, p. 472). Accordingly, the excessive serotonin release would be expected to result in excessive serotonin degradation and hence depletion (i.e. low levels) of serotonin over time. The observed increased serotonin synthesis capacity in migraineurs would also be consistent with the body's attempt to replenish the depleted serotonin levels.

Clinical Corroboration—Primary Dysmenorrhea:

Women with primary dysmenorrhea have increased contractility and increased frequency of contractions of the uterine muscle. Prostaglandins are released during menstruation due to destruction of the endometrial cells and prostaglandin levels are higher in women with severe menstrual pain. Prostaglandin inhibitors such as NSAIDs can provide relief.

It should be noted that prostaglandins exhibit PTH-like (parathyroid hormone) effects that result in calcium mobilization from the bone (Therapy of Renal Diseases and Related Disorders, Second Edition, Kluwer Academic Publishers, 1991, page 98) and prostaglandin synthetase inhibitors are a textbook method for reducing calcium levels in management of hypercalcemia (Therapy of Renal Diseases and Related Disorders, Second Edition, Kluwer Academic Publishers, 1991, page 98). The increased uterine muscle activity is consistent with the elevated Ca²⁺ nerve hypersensitization and muscle hyper contractility pathways previously presented. The difference is that the primary source of the elevated extracellular calcium is likely of prostaglandin-osteoclast origin, rather than of estrogen-osteoclast origin. However, the use of osteoclast downregulation methods of present invention would still have applicability as a treatment option (i.e. regardless of whether an estrogen drop or prostaglandin rise was behind the osteoclast upregulation/calcium release).

Other Corroboration—Susceptibility to Underlying Etiology

Magnesium: Magnesium deficiency is observed in 45% of women with menstrual migraine (Anita H. Clayton, M D, “Menstrual Migraine”, Primary Psychiatry, 2006). This is consistent with the pathogenesis of present invention, as magnesium (Mg²⁺) is a physiological calcium (Ca²⁺) channel blocker (R. Loch Macdonald, “Cerebral Vasospasm” Thieme, 2005, p. 43). As a calcium channel blocker, magnesium would function antagonistically to Ca²⁺ channel mediated effects such as enhanced neurotransmitter release and enhanced muscle contraction activity, previously disclosed. In relation to cerebral vasoconstriction, “hypo-magnesemia increases both calcium uptake and calcium release from the sarcoplasmic reticulum, causing vascular smooth muscle contraction” (R. Loch Macdonald, “Cerebral Vasospasm” Thieme, 2005, p. 44). In context of present invention, patients with low magnesium levels would be at a disadvantage in offsetting the “calcium spike” and hence more susceptible to the physiological effects that are mediated by Ca²⁺ channels.

Low Calcium Levels During Luteal Phase: U.S. Pat. No. 6,228,849 ('849) for PMS treatment methods discloses that “women with PMS had significantly lower calcium levels during the luteal phase of the menstrual cycle” (Col. 1, lines 47-49) and '849 claims administration of calcium and vitamin D as a treatment method for PMS. The observation of lower calcium levels during the luteal phase is consistent with pathogenesis of present invention as the luteal phase is when the estrogen levels are highest and calcium “reservoiring” occurs as shown in FIG. 2. It is the period when calcium would be removed from circulation and stored in bone, and lower calcium levels could be indicative of more aggressive reservoiring in some women, which could then be expected to result in more aggressive calcium release when the estrogen levels drop. It should be noted that '849 deals with a much broader timeline in the ovulation cycle (“mood and behavioral disturbances in women during the latter half of their menstrual cycle”) versus present invention which only includes the short time period around the start of menstruation. Moreover, present invention proposes treatments exactly opposite to treatments to '849, not only curtailing calcium and vitamin D intake during the premenstrual time period of present invention, but also taking aggressive actions to reduce extracellular calcium levels during this time period. Prior art patent '849 also embodies prior art's observation/symptom based treatment approach, versus present invention's etiology based treatment approach, which in part leads to present invention's exactly opposite treatment methods, as present invention's underlying etiology identifies a completely different set of circumstances at the beginning of the “latter half of their menstrual cycle” versus the end of the “latter half of their menstrual cycle”. However, certain underlying observations of '849 are useful in that they are consistent with pathogenesis of present invention, and potentially identify another factor for exacerbated underlying etiology in certain patients (i.e. aggressive calcium reservoiring during the luteal phase eventually leads to higher calcium release prior to menstruation).

Hypothyroid: It should be noted that although current prior art literature does not consider thyroid function in premenstrual headaches or primary dysmenorrhea, we were able to find reference to studies related to thyroid function and PMS (Moline and Zendell, “Evaluating and Managing Premenstrual Syndrome”, 2000, Medscape). In the 1980's, a research group found that 90% of their patients with PMS had 1 or more symptoms of hypothyroidism. However, a double blind study that administered levothyroxine (thyroxine), the major hormone secreted by the thyroid that controls the rate of metabolic processes, did not show any benefit over the placebo. The disappointing results apparently killed further research in this area. The present invention does not focus on thyroxine, but instead focuses on another thyroid hormone, calcitonin. Hypo production of calcitonin, the major hormone used for calcium level downregulation, would impair the body's ability to manage the elevating systemic calcium levels and eventual “calcium spike” in a timely manner. Hypo production of calcitonin would be expected to result in elevated Ca²⁺ levels during the “calcium spike” and is consistent with the underlying pathogenesis presented.

### Methods of Present Invention

In general, methods of present invention employ a simple philosophy: Targeting the underlying etiology is best, targeting the earliest downstream event(s) in the pathway is second best, and targeting further downstream events is least desirable.

Accordingly, present invention proposes to modulate osteoclast activity, and/or directly modulate Ca²⁺ levels, to counteract or “temper” the release rate of reservoired calcium from the bone during the time period of dropping estrogen levels.

The objective is to replace the sharp, premenstrual, estrogen related Ca²⁺ release with a more moderate transition that prevents the transient extracellular Ca²⁺ spike, in order to prevent the subsequent nerve and muscle hypersensitization.

### Materials of Present Invention

Since methods of present invention are directed toward counteracting the escalation in extracellular Ca²⁺ levels that result during the sharp decline in estrogen levels, any suitable materials or methods that decrease osteoclast population density, decrease osteoclast activity or functionality so as to decrease Ca²⁺ release from bone, decrease Ca²⁺ absorption from the intestines, or decrease Ca²⁺ reabsorption by the kidneys may be used.

Any materials or methods that inhibit activation of sensory receptors (e.g. topical anesthetics such as lidocaine, benzocaine etc . . . , earplugs for auditory hypersensitivity) or inhibit function of hypersensitized neurons (e.g. calcium channel blockers, neuron hyperpolarizing agents, etc . . . ) may be used concurrent with methods of present invention. Methods of reducing dietary intake of calcium or vitamin D, or avoiding exposure to sunlight to prevent synthesis of vitamin D, should also be employed. Methods of expanding extracellular fluid to reduce concentrations of calcium may also be used.

Some representative examples of such materials include, but are not limited, to the following:

Calcitonin:

Calcitonin can be used to inhibit Ca²⁺ release from bone via its inhibitory effects on osteoclasts. Calcitonin causes osteoclast to lose their ruffled border which causes a marked transient inhibition of the bone resorptive process. Calcitonin also causes increased excretion of calcium (and phosphate and sodium) by the kidneys and evidence exists that calcitonin also reduces absorption of calcium in the gastrointestinal tract. Calcitonin is available in injectable form (e.g. Calcimar from Rhone-Poulenc Rorer or Caltine from Ferring) or as a nasal spray from (e.g. Fortical from Upsher-Smith or Miacalcin from Novartis) and oral formulations are currently under development. Calcitonin salmon is typically used (because of its greater potency), however because of the potential for allergic reactions adequate precautions should be taken as outlined in the prescribing information. Injections of 4-8 IU/kg (IM or SubQ) drop serum calcium levels by 1-2 mg/dl in most patients. Nasal administration of 2 IU/kg of salmon calcitonin results in a peak reduction of around 5% after 30 minutes of administration with an overall reduction in serum calcium of around 3.2% as expressed as the net change in AUC over 8 hours. Newer nasal formulations of polyethylene glycol conjugated salmon calcitonin have been able to boost the peak serum level reduction to 13% with an overall AUC reduction of 11.9%. Nasal calcitonin-salmon sprays (Miacalcin from Novartis and Fortical from Upsher-Smith) deliver 200 IU per spray and contain sufficient mediation of around 30 such doses. They are also fairly safe, as both Miacalcin and Fortical were tested at single 1,600 unit doses, and doses of 800 IU per day for 3 days, without serious adverse events.

Phosphate:

Either oral or intravenous phosphate is effective in reducing serum calcium levels by causing a shift of calcium out of the extracellular fluid into bone and bone resorption is also inhibited (Therapy of Renal Diseases and Related Disorders, Second Edition, Kluwer Academic Publishers, 1991, page 96). Phosphate precipitates calcium to form calcium phosphate. Phosphate may also increase the efficacy of calcitonin therapy, since calcitonin increases renal clearance of phosphate, thereby attenuating its own effectiveness via this pathway when used alone (Therapy of Renal Diseases and Related Disorders, Second Edition, Kluwer Academic Publishers, 1991, page 97). Daily doses of 1-3 g of elemental phosphorus in three divided doses are typically used for management of hypercalcemia and therapy is contraindicated in renal failure and in the presence of serum phosphorous levels above 5 mg/dL. For purposes of present invention, doses would likely be started at the lower level of 1 g elemental phosphorus and escalated if necessary.

Estradiol:

Exogenous estrogen can be used transiently, at very low doses, during the period of the most precipitous estrogen drop in order to temper the estrogen drop related calcium spike. A study (Ginsburg et. al., “Half life of Estradiol in Postmenopausal Women”, Gynecologic and Obstetric Investigation, 1998;45.45-48) showed that a 0.10 mg estradiol transdermal patch for thirteen hours resulted in an escalation of serum estrogen levels from a baseline of 19 pg/ml to 112 pg/ml and the mean half life of estradiol after removal of a transdermal patch was 2.7 hours (which puts the terminal half life at around 9 hours). The 112 pg/ml (0.1 ng/ml) is around the baseline level of estrogen levels during menstruation and could be used to cushion the decline in estrogen by administration of the patch on days 26-28 of the menstrual cycle when the estrogen decline is steepest as shown if FIG. 1. The dose is low enough and for a short enough period of time that it should not materially interfere with any of the menstrual processes. Alternatively, the patch may also be cut in half to obtain a half dose (0.05 ng/ml) cushion factor. Estrogen is available in oral, injectable, and patch forms from numerous suppliers and include Estrace, Cenestin, Enjuvia, Femtrace, Gynodiol, Menest etc . . . . An estradiol patch is used in preferred embodiment of present invention (e.g. Climara from Bayer) because of the continuous estradiol delivery (hence constant osteoclast inhibition) and because of the short terminal half life after removal of the patch. Likewise, androgens, such as testosterone, also inhibit osteoclast activity, and could be substituted.

SERM:

Selective estrogen receptor modulators (SERMs) such as raloxifene hydrochloride (Eli Lilly's Evista) or generically available tamoxifen, that bind competitively to estrogen receptors and have estrogen-like effects on osteoblasts/osteoclasts and decreases resorption of bone, but lack estrogen-like effects on uterine and breast tissue, would be preferred. Eli Lilly's Evista comes in 60 mg tablets. A single dose elevates serum concentrations to 0.5 ng/mL for every mg/kg of dose and has a serum elimination half life of 28 hours. Multiple dose administration results in maximum serum concentrations of 1.36 ng/mil with a serum elimination half life of 32.5 hours.

Bisphosphonates:

Bisphosphonates can be used to inhibit osteoclastic activity and induce osteoclast apoptosis, however their use is somewhat more tricky because of their potency and longer lasting effect. Bisphosphonates include pamidronate, clodronate, zoledronate, etidronate, alendronate, risedronate, tiludronate, ibandronate, YH 529, EB-1053, incadronate, olpadronate, and neridronate. Although the newer generation bisphosphonate have much more potency and longer terminal half lives, the older generation bisphosphonates with shorter functional half lives are better suited to needs of present invention. As a representative example, a 7.5 mg/kg dose of etidronate disodium (2 h infusion, X3 days) was able to drop serum calcium level by 2 mg/dl (from a baseline of 13.8 mg/dl) in 3 days and maintain efficacy for more than a week thereafter. Accordingly, for purposes of present invention the dose used would be less than half of the above dose (or alternatively a single infusion) and would need to be started around the start of the natural estrogen drop (around day 21 per FIG. 1) so that the ramp up time of the drug matched the ramp down trend of the estrogen and maintained a modest degree of efficacy through the decline period (and possible through the menstruation period for patients with primary dysmenorrhea).

Vitamin D:

Downregulation of Vitamin D by avoidance of sunlight and dietary restrictions should be used to inhibit upregulation of osteoclast activity by inhibiting the VDR receptor pathway previously disclosed.

Other:

The above are only a few representative examples of materials that can be used to modulate cancer progression via modulation of osteoclast activity and are presented only in order to fulfill the reduction to practice requirements of present invention and are not intended to limit the scope of present invention as any suitable compounds may be use instead of, or in combination with, the compounds disclosed above. Saline hydration is often used to reduce calcium levels, either alone or with another therapy. Numerous other agents are also used or under development. As an example, Novartis is currently developing AAE581, a Cathepsin K inhibitor, which specifically inhibits the most potent enzyme involved in bone resorption, and accordingly could be used. As another example, gallium containing compounds can also be used to inhibit osteoclast activity. Another example is selective inhibitors of osteoclast vacuolar proton ATPase, which inhibit osteoclast activity. Another example is integrin receptor antagonists, which inhibit bone resorption by inhibiting integrin in osteoclasts, which is crucial for osteoclast cytoskeletal organization, cell migration, and cell polarization. Other examples would include PTH antibodies.

Adjuvants:

The above may also be used with adjuvants used under prior art to manage hypercalcemia, with doses adjusted accordingly to avoid hypocalcemia or other ill effects. Representative examples of prior art methods include, but are not limited to, expansion of extracellular fluid by administration of sodium solution (either IV or oral increased ingestion of water and salt or commercially available electrolyte solutions which typically contain sodium, potassium, and chloride), use of loop diuretics such as furosemide, bumetanide, ethacrynic acid, and torsemide that inhibit calcium reabsorption in the kidney and glucocorticoids such as prednisone. Calcium channel blockers may also be used and include over the counter compounds such as magnesium (e.g. 1 g/day) to non over the counter calcium channel blockers such as nimodipine or nicardipine. Calcium channel blockers would inhibit the calcium channel mediated excessive neurotransmitter release at nerve synapses and inhibit the increased muscle contractility from calcium channel mediated release of excessive amount of calcium into muscle fibers.

### Assay Materials and Methods

Various prior art methods may be used to hone the doses used and the timing of drug administrations relative to the menstrual cycle or menstrual symptoms. Any suitable prior art materials and methods for monitoring estrogen levels, osteoclast activity, or serum calcium concentration may be used or any suitable materials and methods for determining non bone resident calcium concentrations may be used.

Various materials and methods exist under prior art and representative examples of the above include, but are not limited to the following:

Monitoring estrogen levels would be useful for present invention and blood, saliva, and urine estradiol tests are available under prior art. Home saliva tests such as FemaleCheck provide a convenient method of tracking estrogen levels over the ovulation/menstruation cycle. Alternatively, estradiol hormone levels can be approximated for women that have regular menstrual cycles by use of an ovulation cycle diary.

Since methods of present invention modulate osteoclast activity and calcium levels, any suitable prior art assays and tests may be used to monitor progress or insure safety. The target of present invention is reduction of osteoclast activity and extracellular calcium levels. Calcium blood levels may not accurately represent extracellular levels (e.g. blood concentrations are more tightly controlled and have the advantage of renal clearance, unlike extracellular levels). Monitoring serum levels is more relevant for safety purposes. Normal levels of serum calcium are in the range of 8.0 to 10.8 mg/dl (2.2 to 2.7 mmol/L) and the ionized calcium normal range is approximately 4 to 4.9 mg/dl and serum levels have more relevance for safety purposes by insuring the lower limits are not exceeded when using methods of present invention. Prior art methods may be used to observe efficacy of osteoclast downregulation over time by observing markers of osteoclast activity. Simple methods for determining osteoclast activity include measurement of various protein fragments and minerals released into the blood by the bone dissolving activity of osteoclasts (i.e. serum biochemical markers of bone resorption rates) such as calcium, deoxypyridinoline (DPD), or bone-specific alkaline phosphatase (BAP). During bone resorption, bone collagen is degraded, resulting in the release of calcium and several collagen cross links into the blood. Deoxypyridinoline (DPD) is involved in intermolecular and intramolecular cross linking and is specific to bone degradation. Monitoring serum concentrations of calcium (e.g. using s-cresolphthalein complexone; lyatron Co., Tokyo, Japan) or phosphate (e.g. enzyme assay; Kyowa Co., Tokyo Japan) are also available methods, as both are released into blood by bone resorption. Urinary excretion of pyridinoline and deoxypyridinoline, using Osteomark (NTX, Ostex) and Crosslaps (CTX, Osteometer) assays, are other methods available under prior art for monitoring bone resorption.

### REDUCTION TO PRACTICE EXAMPLES

The guiding principle embodied in the examples under present invention is basically transient “osteoclast modulated, calcium modulated” regimens for attenuating the rate of calcium release caused by premenstrual declines in estrogen levels, in order to attenuate the premenstrual/menstrual symptoms that afflict certain patients.

The examples are progressive and for the sake of brevity only the rationale for the incremental changes from the prior examples is discussed in successive examples.

### Example 1

### Premenstrual Headache

Condition: A patient presents with premenstrual headache. The headache starts the day before the start of menstrual bleeding and lasts until the start of menstrual bleeding. The headache first manifests as a low level headache and ramps up over several hours into persistent, intense pain that in not at the very back or very front of the head and is accompanied by a hypersensitivity to sound (but not to light). The headache may be accompanied by nausea and irritability. The sufferer prefers a dark, quiet room and going to sleep, as the headache is gone by the next morning at the start of menstruation. The patient is currently not taking the birth control pill. The patient has irregular periods.

Prior Art Treatment: Under prior art, the patient is instructed to try any of the common nonprescription analgesics (aspirin, acetaminophen, ibuprofen) at the onset of the headache.

Present Invention Treatment: Under present invention, after patient is screened for calcitonin allergies in accordance with the manufacturers prescribing information, the patient is prescribed nasal calcitonin-salmon spray (Miacalcin from Novartis and Fortical from Upsher-Smith) and instructed to administer a spray in each nostril (i.e. 200 IU×2=400 IU) at the earliest signs of onset of headache. The patient is instructed to administer 400 IU (or 200 IU) every 8 hours as needed, up until the start of menstrual bleeding.

In addition, the patient is instructed to curtail calcium and vitamin D intake and to avoid sunlight.

In addition, the patient may be instructed to employ “sensory deprivation” techniques such as applying a topical anesthetic (e.g. lidocaine, benzocaine, etc... ) to the face, hands, and feet If there is any hypersensitivity to sound, wear ear plugs.

The patient may also be instructed to employ one or more prior art methods of hypercalcemia reduction as an adjuvant (e.g. expansion of extracellular fluid, calcium channel blockers, etc . . . ).

Rationale for Calcitonin:

Having previously disclosed the etiology as upregulated osteoclast activity (caused by the drop in estrogen levels) the administration of the more potent, exogenous salmon-calcitonin is intended to inhibit/reduce osteoclast mediated release of calcium during the sharp transition period between “reservoiring of calcium” and “release of calcium” and until the body's own calcitonin regulation system can cope with the condition. Present invention targets the earliest steps of the underlying etiology to prevent the cascade of downstream undesirable events that follow. Present invention's targeting and treating the underlying cause of the condition is in contrast to prior art's treatment of symptoms of the condition.

Rationale for Curtailed Calcium and Vitamin D Intake:

Ingested calcium would elevate calcium levels and reduce the benefit gained from calcitonin's downregulation of osteoclasts and extracellular calcium. Vitamin D is also antagonistic to calcitonin, as it upregulates osteoclasts via VDR receptors as previously disclosed, increases gastrointestinal absorption of calcium (via enhancing synthesis of the cytosolic calcium-binding protein CaBP, which transports Ca²⁺ from the mucosal to serosal cells of the gut), and has been implicated in stimulating reabsorption of calcium by the kidneys, while promoting the excretion of phosphate. Accordingly, all patients with premenstrual headaches should curtail calcium intake and vitamin D intake, as both would exacerbate the underlying etiology, and in the case of present invention could potentially preclude the calcitonin treatments from functioning as envisioned.

Rationale for Sensory Deprivation:

If sufficient attenuation of the extracellular calcium levels cannot be achieved by calcitonin, the topical anesthetics and ear plugs are effectively “sensory deprivation” methods that function to inhibit firing of the “hypersensitized” neurons in the first place. Topical anesthetics that inhibit neuronal transmission are antagonistic to “depolarizing” events, such as escalating extracellular calcium concentration. The choice of hands, feet, and face for application is based on the somatosensory neuronal mapping of FIG. 3b, as those three account for around two thirds of the input into the somatosensory cortex. The earplugs attenuate input into the auditory cortex, also shown in FIG. 3b.

Rationale for Adjuvants:

The faster acting prior art methods of reducing calcium, or counteracting the effects of elevated extracellular calcium, were chosen to provide both fast pain relief as well as better pain relief. If sufficient attenuation of the extracellular calcium levels cannot be achieved by calcitonin, these prior art methods would work synergistically with calcitonin to provide a better treatment method.

### Example 2

Premenstrual Migraine

Condition: A patient presents with premenstrual migraine headaches. The headache starts the day before the start of menstrual bleeding and lasts for 2-3 days. The patient has irregular periods.

Prior Art Treatment: Acute treatment of menstrual migraines includes triptans (serotonin agonists), prostaglandin synthesis inhibitors, or ergotamine tartrate. Nonsteroidal anti-inflammatory drugs have limited efficacy in most women with menstrual migraine.

Present Invention Treatment: Under present invention, the patient is prescribed nasal salmon calcitonin as described in Example 1. The patient is instructed to curtail calcium and vitamin D intake and avoid sunlight for the reasons outlined in Example 1. The patient is also prescribed raloxifene or a low dose of estradiol as outlined below.

Estradiol Patch: As a representative example, the patient is prescribed a 0.05 mg estradiol patch (e.g. Climara from Bayer) to be applied at the earliest sign of migraine and to be kept on for around 3 days. Based on the data previously presented, a 0.05 mg estradiol patch should raise serum estradiol levels about 50 pg/ml (0.05 ng/ml) and would serve to reduce the magnitude of the calcium spike. The estradiol dose is low enough it should not interfere with normal ovulation cycle event, only to modestly temper the abrupt transition period that occurs prior to menstruation, particularly when used concurrent with the calcitonin.

SERM: In the preferred embodiment of present invention, a SERM such as raloxifene is used. Raloxifene inhibits osteoclast activity, which in turn inhibits the rise of extracellular calcium levels. As a representative example, the patient is initially prescribed a 0.5 mg/kg oral daily dose of Raloxifene hydrochloride to be taken at the earliest indications that a migraine will occur. Based on data previously presented, a single dose as prescribed above, would provide a maximum plasma concentration of around 0.25 ng/dl with a serum half life of around 28 hours (versus 3 hours for estradiol). Two or three such daily doses should be adequate to cover the potential migraine period and would provide longer term osteoclast inhibition and reduced extracellular calcium levels. The selectivity of raloxifene's effect to osteoclasts, without uterine effects, allows for more potent mitigation, without the worry about potential interference with menstrual related uterine events.

It should also be noted that the use of raloxifene, particularly at higher doses and for longer duration (e.g. week or so) would allow a larger portion of the “reservoired” calcium to be retained in the bone. Over many menstrual cycles, this could potentially lead to women being able to “bridge the gap” between men, in terms of peak bone mass achieved by age 40 (previously disclosed under the bone micro environment section of this application), and as such potentially lower the women's risk of osteoporosis later in life. Estradiol use in such a manner would not be preferred, as it could potentially interfere with the menstrual cycle, via its interaction with uterine estrogen receptors.

Rationale for Calcitonin/Raloxifene:

The calcitonin inhibits the underlying rise in calcium by inhibiting osteoclast activity, enhancing urinary excretion of calcium, and inhibiting intestinal absorption of calcium. Calcitonin is fast acting but results in a modest decrease in extracellular calcium.

Raloxifene is slower acting but more potent and longer lasting. Selective activation of bone related estrogen pathways would inhibit osteoclast population density by binding to osteoblast receptors and increasing their output of OPG and suppressing their RANKL production as well as mimicking estrogen's other effects such as prolonging lives of osteoblasts while simultaneously promoting osteoclast apoptosis. All of these pathways result in reduced extracellular calcium levels. The inability of the SERM to activate estrogen receptors in breast of uterine tissue would prevent potentially undesirable effects. SERMs provide more targeted activity, which is desirable.

When used concurrently, the calcitonin is fast acting with modest, short duration of effect and the raloxifene is slower acting but has much more potent, longer duration of effect. Together the two would provide dual action, sustained effect over the anticipated course of the migraine. Calcitonin would impair osteoclast functionality/activity by making osteoclasts lose their ruffled “bone dissolving” border and raloxifene would downregulate osteoclasts by population density pathways.

### Example 3

### Premenstrual Migraine

Condition: A patient presents with premenstrual migraine headaches. The headache starts the day before the start of menstrual bleeding and lasts for 2-3 days. The patient has regular periods and keeps tract of dates of anticipated menstruation.

Prior Art Treatment: Acute treatment of menstrual migraines includes triptans (serotonin agonists), prostaglandin synthesis inhibitors, or ergotamine tartrate. Nonsteroidal anti-inflammatory drugs have limited efficacy in most women with menstrual migraine.

Present Invention Treatment: Under present invention, the patient is prescribed a SERM such as raloxifene (covered in Example 1), with the administration of the SERM starting 2 or more days prior to the expected start of menstruation. As a representative example, the patient is initially prescribed a 0.5 mg/kg daily dose of Raloxifene hydrochloride to be taken starting three days prior to the anticipated start of menstrual bleeding (i.e. day 26 on FIG. 2) and ending one or two days after start of menstrual bleeding.

The patient is also prescribed calcitonin (covered in Example 1) to be used in the event the patient should start experiencing any indications that a migraine headache was beginning.

The patient is instructed to curtail calcium and vitamin D intake and avoid sunlight for the reasons outlined in Example 1. Any adjuvants previously described may also be recommended.

Alternatively, the patient may be prescribed a higher dose of the SERM, to be administered for a longer period of time, to provide the additional benefit of preserving the “reservoired calcium”. As a representative example, a 1 mg/kg daily dose of Raloxifene hydrochloride would boost blood levels to 0.5 ng/dl, which is higher than the highest estrogen levels achieved during the ovulation cycle (FIG. 2), and would provide a very high level of osteoclast inhibition and hence calcium retention. Increasing the duration of the regimen (e.g. starting 5 or 6 days prior to start of menstruation, or when estrogen levels first start dropping as shown in FIG. 2) should also result in an increased amount of the “reservoired” calcium being retained in bone. As previously disclosed, this may have advantages for reducing osteoporosis risks later in life.

Innumerable combinations of dose levels, multiple dose administrations, and initiation of treatment earlier than two or three days prior to start of menstrual bleeding may be substituted as indicated. The above are only intended as a few representative examples.

Rationale for Raloxifene/Calcitonin:

The earlier, prophylactic use of Raloxifene in this example (i.e. days prior to anticipated premenstrual migraine) is made possible by the patient's regular period (unlike prior examples where the patient had irregular periods). The calcitonin is provided as a “just in case” option, should the raloxifene be unable to adequately control the calcium spike, or should the patient's intake of calcium or vitamin D, push the calcium levels beyond a desired threshold.

### Example 4

### Premenstrual Headache or Migraine

Condition: A patient presents with premenstrual headaches or premenstrual migraine headaches. The patient is on the birth control pill, and experiences the headaches during the week when the birth control pills do not contain the estrogen/progesterone (i.e. birth control pills contain a combination of estrogen/progesterone for 3 weeks, and a “sugar pill” for 1 week, to allow for menstruation).

Prior Art Treatment: Prior art treatments for premenstrual headaches are summarized in Example 1 and prior art treatment methods for premenstrual migraine headaches are summarized in Example 2.

Present Invention Treatment: Under present invention, a preemptive approach is taken. The pills that do not contain estrogen are substituted with 30 mg raloxifene tablets (or any other suitable dose, or declining dose regimen, or other comparable SERM may be substituted). Alternatively, oral formulations of calcitonin may by be substituted for, or combined with, the SERM formulations that replace the “sugar pills”. Alternatively, calcitonin may be prescribed separately, as a “just in case” treatment in the event a headache should begin to manifest.

In alternate embodiments, any adjuvants may be included, as long as the regimen functions to downregulate osteoclast population density or activity in order to prevent an abrupt elevation in extracellular, non bone resident, calcium concentrations or functions to reduce extracellular calcium concentrations or counteract their physiological effects.

Rationale: Dosing of the “blank” birth control pill in the regimen, using methods of present invention, allows for exceptionally precise timing and control of osteoclast downregulation. The magnitude and duration of the estrogen drop can be predetermined in advance, and adjusted as necessary over time, to effectively prevent the patient from ever having to experience the headaches. The methods of present invention can be used to both prevent the occurrence of headaches in advance, and to retain more of the “reservoired” calcium in bone for potential future benefit.

### Example 5

### Primary Dysmenorrhea

Condition: A patient presents with severe menstrual cramps that start several hours prior to the start of menstrual bleeding and taper off a day or two into menstrual bleeding. The use of NSAIDS is contraindicated because the patient has gastrointestinal bleeding (or other condition that precludes the use of NSAIDs).

Prior Art Treatment: The primary prior art treatment method is administration of prostaglandin inhibitors such as Naproxen (Aleve), Ibuprofen (Advil), and Mefenamic Acid to provide some relief for discomfort.

Present Invention Treatment:

If the patient has irregular periods, she is prescribed nasal calcitonin, to be administered at the earliest signs of onset of cramping and every 8 hours thereafter as needed, but not beyond the end of menstrual bleeding. The patient is also prescribed a 60 mg raloxifene tablet to be taken at the start of cramping.

If the patient has regular periods and can reasonably predict the anticipated start of menstruation, the patient is prescribed a 30 mg raloxifene tablet to be taken daily starting two to three days prior to the anticipated start of menstruation and stopping administration a day after start of menstruation.

If the patient is taking birth control pills, the non estrogen containing pills are replaced with 30 mg raloxifene tablets (or any other suitable dose) for one or more of the 7 days.

The patient is instructed to curtail calcium and vitamin D intake and avoid exposure to sunlight.

The patient may also be instructed to take adjuvants such as calcium channel blockers.

Rationale: Hyperactive uterine contractions could be fueled by either the estrogen-osteoclast related extracellular calcium spike (via the nerve and muscle pathways previously disclosed) or the prostaglandin induced calcium spike (via prostaglandin's PTH-like effects that result in calcium mobilization from the bone, previously disclosed), or both. The osteoclast downregulation methods of present invention would provide therapeutic benefit, regardless of which one it was.

Compounds such as calcitonin or raloxifene would inhibit the release of bone resident calcium from both the estrogen related pathways and the prostaglandin related pathway. Inhibiting osteoclast functionality/activity (calcitonin) and inhibiting osteoclast population density (raloxifene) serve to inhibit both the estrogen-drop related mobilization of calcium form the bone and the prostaglandin mediated mobilization of calcium from bone.

### Example 6

### Migraine Headache

Condition: A patient (either male or female) presents with periodic migraine headaches that include a throbbing pain on one side of the head (trigeminal nerve involvement) and exhibit traditional signs of depolarization in the cortex, and vasoconstriction in the brain.

Under present invention: The patient is prescribed nasal calcitonin (e.g. as in Example 1) and raloxifene (e.g. as in Example 2). The patient is also instructed to curtail calcium and vitamin D intake and may also be prescribed/instructed to take any of the adjuvants previously disclosed for managing hypercalcemia (e.g. expansion of extracellular fluid, calcium channel blockers etc . . . ).

Rationale: The symptoms described in this example are all consistent with the underlying pathways of elevated extracellular calcium concentrations, as presented in this application. The source of the elevated calcium may be endocrine/osteoclast modulated, prostaglandin modulated, or from any other cause. The cause of the elevated calcium is irrelevant to treatment methods of present invention.

Moreover, it is not even necessary for the underlying etiology to be related to elevated extracellular calcium levels for methods of present invention to provide therapeutic benefit. This can best be explained by following the pathways previously presented in reverse, showing how decreasing extracellular calcium would provide therapeutic benefit in the above migraine, even if elevated extracellular was not the dominant underlying etiology.

Irritation of Trigeminal Nerve: Reducing extracellular calcium concentrations would reduce hypersensitization of the trigeminal nerve by going in the reverse direction of the neuronal hypersensitization pathways previously presented. First, reducing extracellular calcium concentrations would “hyperpolarize” the neuronal membrane via the Nernst equation previously presented, meaning that a much larger input signal would be required to depolarized the neuron to the point where it crosses threshold potential and initiates the “all-or-none” signal. Second, the reduced calcium concentrations would result in less calcium entering through the voltage gated calcium channels (i.e. as both the absolute extracellular concentrations and ratio of extracellular to intracellular concentrations would be smaller) and hence less synaptic neurotransmitter release would result. The lower inrush of calcium would also be more readily cleared to prevent any PTP type effects. Accordingly, the trigeminal nerve would be “desensitized” by lowering extracellular calcium concentrations, in an exactly opposite manner by which it is “hypersensitized” during rising extracellular calcium concentrations. The use of adjuvants such as calcium channel blockers (e.g. magnesium) would contribute to further “desensitizing” the trigeminal nerve by further inhibiting neurotransmitter release at the synapse.

Cortex Depolarization: The same 3 neuronal “desensitization” mechanism described above (i.e. neuronal membrane hyperpolarization, decreased neurotransmitter release, and reduced mP) would also apply to neurons in the brain, and provide therapeutic relief.

Vasoconstriction: Reducing extracellular calcium levels would reduce muscular contractions, as can be seen by going in reverse down the neuromuscular pathways previously presented. First, the reduced calcium concentrations would result in less calcium entering through the voltage gated calcium channels at the neuromuscular junction (i.e. as both the absolute extracellular concentrations and ratio of extracellular to intracellular concentrations would be smaller) and hence less synaptic release of acetylcholine would result ( reducing the level of the traveling depolarization over the surface of the muscle tissue). Second, the reduced calcium release into muscle fibers (also via voltage gated calcium channels) would reduce the level of tropomyosin block removal and hence reduce the level of actin-myosin cross bridging. This would weaken the muscle contractions. Adjuvants such as calcium channel blockers (e.g. magnesium) would further enhance the effect by inhibiting calcium signaling responsible for both neurotransmitter release at the neuromuscular junction and inhibit Ca²⁺ release into muscle fibers, as both function by calcium signaling through calcium channels.

Accordingly, present invention's methods of reducing extracellular concentrations of calcium would provide therapeutic benefit by attenuating the symptoms/observations related to migraines, regardless of whether or not elevated extracellular calcium was part of the underlying etiology.

### Example 7

### Other Headaches

Other headaches that have symptoms that indicate they could benefit from nerve and muscle desensitization by methods of present invention include hangover headaches, headaches from extreme exercise, and muscle tension headaches. As a representative example, hangover headaches are presented below to convey how the pathways disclosed under present invention are applicable.

Hangover Headaches: After excessive drinking, a male patient has an alcohol related headache and is sensitive to loud noises. Drinking large amounts of water helps reduce the headache.

These symptoms are consistent with the Ca²⁺ nerve hypersensitization pathways previously presented. The distribution of the headache is consistent with the somatosensory cortex mapping. Sensitivity to loud noises is consistent with auditory hypersensitization, and less severe than the auditory hypersensitivity observed in a premenstrual headache. Drinking large amounts of water is a way of reducing Ca²⁺ concentrations by expanding extracellular volume.

There is also a compelling case for elevated extracellular Ca²⁺ levels being part of the underlying etiology. The elevated Ca²⁺ would be expected from both prostaglandin-osteoclast and endocrine hormone-osteoclast interactions.

First, alcohol kills cells (i.e. denatures and precipitates proteins, extracts cholesterol and other lipids). Dead cells cause prostaglandin release. Prostaglandins have PTH like effects that result in release of calcium from bone.

Second, alcohol cases a drop in testosterone levels (although some individuals may experience a transient rise in testosterone level during intoxication, according to recent studies). Testosterone is aromatized into estrogen to achieve osteoclast inhibition, and some studies have also shown a direct effect of testosterone on osteoclast inhibition. A drop in testosterone would release the inhibitory effect on osteoclasts, resulting in release of calcium form the bone.

NSAIDs that function to inhibit prostaglandins are generally effective for these types of headaches. However, for patients where NSAID use in contraindicated, methods of present invention may provide an alternative treatment option. As an example, the patient may take a raloxifene tablet well in advance of the anticipated headache. As another example, the patient may also take nasal calcitonin prior to or upon emergence of the headache. Methods of present invention inhibit both sources of calcium elevation (i.e. prostaglandin mediated and testosterone level mediated).

As in Example 6, for these headaches, the treatments of present invention will target the underling etiology, in whole or in parl Otherwise, treatment of present invention will target symptoms, which is consistent with prior art practice, however present invention will target novel pathways.

### Scope of Invention/Alternate Examples

The above representative examples have innumerable variants and are not intended to limit the scope of the invention. The scope of the invention is intended to encompass the following:

1) any premenstrual condition resulting from estrogen-osteoclast mediated elevations in extracellular calcium concentrations or prostaglandin-osteoclast mediated elevations in extracellular calcium concentrations, or both, and

2) any type of headache, in either men or women, that would benefit from reduced extracellular Ca²⁺ concentrations.

The doses, drugs, routes of administration, and adjuvants used in the above representative examples have innumerable variants and are not intended to limit the scope of the invention. The representative examples are not intended to suggest optimal doses, drugs, routes of administration or regimens but only to provide a few representative examples of efficacious and safe treatments to fulfill the reduction to practice requirement of this application. Optimal doses, drugs, routes of administration, and regimens would be further honed as is customary under prior art in controlled human clinical trials.

For purposes of present invention and its related claims, calcitonin is defined as calcitonin, calcitonin agonists, calcitonin analogs, or any molecule that exhibits the biological function of calcitonin or activates pathways normally activated by calcitonin, such as osteoclast activity downregulation, increased renal reabsorption of calcium, or increased absorption of calcium from the gastrointestinal tract. Representative examples of calcitonin include, but are not limited to, human calcitonin, salmon calcitonin, and synthetic salmon calcitonin such as Fortical from Upsher-Smith and Miacalcin form Novartis.

For purposes of present invention and its related claims, anti-osteoclast SERM (selective estrogen receptor modulator) is defined as any molecule that activates pathways normally activated by estrogen, as they relate to osteoclast population downregulation or osteoclast activity downregulation and downregulation of extracellular concentration of calcium. Representative examples of anti-osteoclast SERMs that downregulate osteoclasts include, but are not limited to, raloxifene and tamoxifen.

For purposes of present invention and its related claims, when the word “or” is used, it is used to mean “either or both”.

The scope of invention is intended to encompass the use of any anti-osteoclast compound(s), and not limited to the few representative examples presented. Anti-osteoclast compounds are hereby defined as any substance, either currently known or to be discovered or developed in the future, that inhibit osteoclast related release of calcium from the bone.

The scope of invention is also intended to encompass the use of any adjuvant compounds or methods that function to counteract any aspects of the underlying etiology, or downstream events, as disclosed by present application or that could reasonably be anticipated by one skilled in the art.

### Summary of Novelty and Unobviousness

Prior art has admitted its inability to elucidate the underlying etiology/pathogenesis related to the premenstrual symptoms and related conditions, including premenstrual headaches, premenstrual migraines, and primary dysmenorrhea. Present invention has finally elucidated the underlying etiology/pathogenesis, which is very different from anything even postulated under prior art.

Present invention not only outlined the etiology and subsequent pathways, but corroborated them by explaining the numerous prior art clinical observations in light of the new etiology/pathophysiology.

Because the protocols of present invention are based on a novel etiology/pathogenesis that is not known (and not obvious) to prior art, the treatments presented herein to address the novel underlying etiology/pathogenesis would have also been unobvious to prior art practitioners.

Osteoclast modulation for premenstrual headaches, premenstrual migraines, and primary dysmenorrhea is not a prescribed treatment under prior art, making a further prima facie case for unobviousness.

### Utility

The underlying philosophy is that targeting the underlying etiology is best (most potent), targeting the earliest downstream event(s) in the pathway is second best, and targeting further downstream events is least desirable.

Having elucidated the underlying etiology and subsequent pathways in this application (i.e. from bone to brain, with a lot of pathways in-between), present invention targets the earliest possible events (i.e. in the bone) in order to prevent the subsequent systemic events that result. In contrast, prior art treatments focus on fairly downstream events (i.e. in the brain), in large part because prior art had not identified the underlying etiology and was left with treating symptoms/observations.

Accordingly, present invention will provide great utility by targeting the earliest events in these conditions, which should provide the greatest benefit. Alternatively, combining the etiology based treatments of present invention, with prior art symptoms based treatments as adjuvants, would also greatly improve the potential for pain relief for patients.

1-4. (canceled)

5. A method of treating headaches, including migraine headaches, comprising administration of compound or compounds, in therapeutically effective amounts to inhibit osteoclast activity, functionality, or population density.

6. The method of claim 5 wherein said compound or compounds is calcitonin or raloxifene or both.

7. A method of treating headaches and migraines comprising administration of compound or compounds, in therapeutically effective amounts to reduce concentrations of non bone resident, extracellular calcium ions.

8. The method of claim 7 wherein said compound or compounds is calcitonin or raloxifene or both.
